(19) United States
(12) Patent Application Publication
(10) Pub. No.: US 2006/0200050 A1
(43) Pub. Date: Sep. 7, 2006

(54) Illumination timing device

(76) Inventor: Michael Dobbs, Pebble Beach, CA (US)

Correspondence Address: US CORPORATIONS, INC., 630 NEPTUNE AVE., ENCINITAS, CA, 92024, US

(21) Appl. No.: 11/069,189

(22) Filed: Mar. 1, 2005

(51) Int. Cl. A61B 5/103

(52) U.S. Cl. 600/590

(57) ABSTRACT

An illuminating timing device comprised of an integrated base unit and software, three pedal position foot controls, standard printer, and a re-printable template capable of providing an economical means of performing periodontal depth charting quickly and easily by one person with minimal risk of contamination.

### CROSS-REFERENCE TO RELATED APPLICATION

Not applicable

### STATEMENT REGARDING FEDERALLY SPONSORED RESEARCH OR DEVELOPMENT

Not applicable

### REFERENCE TO SEQUENCE LISTING

Not applicable

### BACKGROUND OF THE INVENTION

This invention relates to a small, self contained, battery/photocell powered, programmable timing device which can be programmed to give a visual/flashing LED indication of the need for attention to the device which the device is attached

Any number of systems operating at a plurality of remote sites may be monitored using only one sensor at the remote sites. However, the device is best used in monitoring any one piece of equipment with one device.

The parameters selected for monitoring are chosen according to their importance in evaluating the operational condition of a system. Many systems such as an elevator system may have typical built in sensors including, among others, an alarm button sensor, a door fully opened sensor, a leveling sensor, a demand sensor, and a brake fully engaged sensor. These sensors produce signals which may be multiplexed into a transmitter for transmittal to a local office which monitors the status of the plurality of elevator systems

The current device allows for equipment without such built in sensors to be monitored for routine maintenance or the replacement of parts.

The invention could be attached to any device that needs regular scheduled maintenance/inspections performed on it.

The device would help alert the user that the equipment needs to have its scheduled maintenance/inspection. The user would be alerted and could then remove it from service and call the engineering/maintenance dept. Thus reducing possible liability/safety issues with using equipment, which is overdue for inspection.

The main application would be for attachment to medical equipment, but it could be used on anything that has scheduled maintenance performed on it.

### SUMMARY OF THE INVENTION

1) From the patent search performed and from our own searches on the USPTO website, we cannot find any one patent that closely approximates our product. The device is simple in construction as in design.

The device is a small, self contained, battery/photocell powered, integrated programmable timing unit which can be programmed to give a visual/flashing LED or similar indication of the need for attention to the equipment, in which attached. The device would be attached by double sticky tape or another simple non-invasive manner to the top or the side of a piece of equipment. The time interval can be adjusted to different lengths of time in days, or a target date in which to alarm and start the flashing LED or signal light.

The current device is primarily designed as a tool for maintaining professional equipment such as found in the medical field. It is designed to aid in the function and maintenance of such day to day operations of such equipment. We consider the end product to have four major integrated components: A standard LED signal light, a calendar, a timer and an interactive programming chip.

Thus, it is an object of the invention to provide an improved illumination timing device.

A further object of the invention is to provide an illuminating timing device construction which is rugged, economical, easily programmed and user friendly mounting system according to the various combinations of equipment panels and exteriors.

Another object of the invention is to provide an improved illuminating timing device construction which includes a body, calendar and timing chip, LED light and programming panel easily operated by the user.

Accordingly, it is an object of the present invention to provide an illuminating timing device which embodies a single uni-body construction.

Further still, it is an object to provide such an illuminating timing device which will function without the use of outside power.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a new illuminating timing device apparatus and which has many of the advantages of the devices mentioned heretofore and many novel features that result in an illuminating timing device which is not anticipated, rendered obvious, suggested, or even implied by any of the prior art tool guides, either alone or in any combination thereof.

It is another object of the present invention to provide a new illuminating timing device which may be easily and efficiently manufactured and marketed.

It is a further object of the present invention to provide a new illuminating timing device which is of a durable and reliable construction.

An even further object of the present invention is to provide a new illuminating timing device which is susceptible of a low cost of manufacture with regard to both materials and labor, and which accordingly is then susceptible of low prices of sale to the consuming public, thereby making such illuminating timing devices economically available to the buying public.

Still yet another object of the present invention is to provide a new illuminating timing device which provides in the apparatuses and methods of the prior art some of the advantages thereof, while simultaneously overcoming some of the disadvantages normally associated therewith.

Yet another object of the present invention is to provide a new illuminating timing device which includes a one-sided fastening device.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### ILLLUSTRATIONS

Not applicable.

We claim:

1. A self contained illumination timing to be placed on functional or operational equipment of any type (primarily health related) which may require regularly scheduled maintenance or attention above and beyond its day to day functions.

2. An illumination timing device according to claim 1 wherein the device embodies a programming interface accessible from the outside in order to program the alarm.

3. An illumination timing device according to claim 1 wherein the device contains a programming device to activate an LED alarm during any pre-determined period.

4. An illumination timing device according to claim 1 wherein the device contains an LED element which is activated by the program to indicate the need for service.

5. An illumination timing device according to claim 1 wherein the device embodies a self contained power source.

6. A illumination timing device according to claim 1 wherein the device is small enough to be supported and attached with adhesive tapes

7. A illumination timing device according to claim 1 wherein the device components on the top of the main unit are sealed by an alcohol resistant plastic label so the unit can be easily wiped clean to disinfect, should they need to touch the main unit

8. A illumination timing device according to claim 1 wherein the device software is designed to minimize the time necessary to record data, automatically incrementing or decrementing the tooth number. teeth, stored on the facial side, are automatically incremented or decremented past on the lingual side.

9. An illumination timing device according to claim 1 wherein the device software allows for the operator to switch between the three recording modes, for example, to record the pocket depth for the entire top half of the mouth and selected teeth on the bottom, further enhancing the speed in which a desired charting can be accomplished.
